(19) United States
(12) Patent Application Publication
(10) Pub. No.: US 2015/0153323 A1
(43) Pub. Date: Jun. 4, 2015

(54) PLASMA SEPARATION SYSTEM AND METHOD FOR PLASMA SEPARATION

(71) Applicant: Roche Diagnostics Operations, Inc., Indianapolis, IN (US)

(72) Inventor: Herfried Huemer, Feldbach (AT)

(21) Appl. No.: 14/615,594

(22) Filed: Feb. 6, 2015

Related U.S. Application Data
(63) Continuation of application No. PCT/EP2013/066529, filed on Aug. 7, 2013.

(30) Foreign Application Priority Data
Aug. 9, 2012 (EP) 12179895.3

(51) Int. Cl. G01N 33/49; B01L 3/00

(52) U.S. Cl. CPC G01N 33/491; B01L 3/502723; B01L 3/5635; B01L 2200/026; B01L 2200/146; B01L 2200/0684; B01L 2300/0681; B01L 2300/14; B01L 2400/049

(57) ABSTRACT

The disclosure relates to a system for separating plasma from whole blood, with a collector vessel for receiving whole blood, a filtering device for extracting plasma, whose input side can be attached to the collector vessel, and with a transport unit furnished with a pump for creating a partial vacuum. The transport unit is part of an analyser and the filtering device together with the attached collector vessel may be docked onto the sample input part of the analyser via the output side of the filtering device.

### CROSS-REFERENCE TO RELATED APPLICATIONS

This application is a continuation of International Patent Application No. PCT/EP2013/066529, filed 7 Aug. 2013, which claims the benefit of European Patent Application No. 12179895.3 filed 9 Aug. 2012, the disclosures of which are hereby incorporated herein by reference in their entirety.

### BACKGROUND

The present disclosure relates to a system for separating plasma from whole blood, with a collector vessel for receiving whole blood, a filtering device for extracting plasma, whose input side can be attached to the collector vessel, and is with a transport unit furnished with a pump for creating a partial vacuum. The disclosure further relates to a filtering device and to a method for separating plasma from whole blood.

Besides centrifuges, which are used mainly in laboratories for separating plasma from whole blood, there are known a number of devices for obtaining small amounts of plasma at decentralized Point of Care (PoC) locations by separating plasma from whole blood by means of filtering.

In the simplest case plasma separation may be effected by means of a multilayer test strip as described in U.S. Pat. No. 5,262,067 A (BOEHRINGER MANNHEIM), where a transport layer on an inert carrier layer is provided for transporting sample fluid (whole blood) from an input area to a measuring area. The transport layer may for instance be made of a glass fibre mat, which in the input area is covered by a plasma separation layer.

From EP 0 550 950 A2 (SANWA KAGAKU KENKYUSHO) there is known a method and a device for separating blood serum and plasma. This document presents diverse variants of devices for plasma extraction, where for instance in FIGS. 1 to 4 variants are described in which a plasma separating device is integrated in a blood sampling device. By means of a partial vacuum blood is first sucked into a collector vessel in which there is disposed a two-layer separating filter. After the blood sample has been taken the collector vessel is connected to an evacuated fluid container, the plasma being separated by the separating filter and collected in the fluid container. In the variant shown in FIGS. 5 and 6 the partial vacuum required for plasma separation is generated by means of a plunger syringe. The variant of FIGS. 9 and 10 furthermore shows a kind of syringe input filter, which may also be used for obtaining plasma.

From WO 2011/033000 A2 (F. HOFFMANN LA ROCHE AG) a multi-part sample input device for entering liquid samples into an analyser has become known. The input device has fittings for establishing a connection between a is collector vessel receiving the sample (for instance a syringe) and the input opening of an analyser. The analyser connector part of the input device has a retaining element (clot catcher) inside, for instance a grid, which keeps particulate components of the sample from entering the analyser. A clot catcher can not be used for separating plasma from whole blood. A sample vessel connector part of the input device, which is permanently attached to the analyser connector part by means of a snap-on connection, has an aspirating tube extending into the interior of the syringe, the fitting of the sample vessel connector part, which is configured as a Luer cone, having venting channels for introducing air into the interior of the syringe during sample taking.

WO 2012/062651 A1 discloses a device for filtration of a liquid blood sample comprising a carrier forming a fluidic system, wherein a separating structure for the blood sample is arranged on a first area of the carrier and a conveyor structure for sample transport is situated at a second area of the carrier separated from the separating device. A positive pressure or a negative pressure can be generated by a manually operated membrane of the conveyor structure in order to expedite or aid the filtration. The uncontrolled pressure values met at the filter unit of the separation structure when pressure is applied manually to the membrane are disadvantageous.

From WO 96/24425 A1 (FIRST MEDICAL INC.), especially from its FIGS. 1 to 3 and 8, a method and device for plasma separation is known. A device called “Blood Separation Device” comprises a filter element, a flexible tube and at its end a needle which is introduced into a “Blood Collection Device”. By means of a motor unit comprising a peristaltic pump acting on the flexible tube whole blood is sucked from the “Blood Collection Device” and pumped through the filter element, whereby plasma is separated and can be obtained for further use at a plasma output opening of the filter unit. The relatively high uncontrolled pressure values met at the filter unit when pressure is applied and the partial vacuum occurring in the collection vessel when whole blood is continuously sucked off are disadvantageous.

Thus, there is a need in the art for a system for separating plasma from whole blood, a corresponding filtering device and a method for separating plasma from whole blood, which should be simple and economical and where reproducible plasma samples may be obtained even from small blood samples and/or samples with high haematocrit values.

### SUMMARY

It is against the above background that the present disclosure provides certain unobvious advantages and advancements over the prior art. In particular, the inventors have recognized a need for improvements in plasma separation systems and methods for plasma separation.

In accordance with one embodiment of the present disclosure, a system is provided where the transport unit is part of an analyser and the filtering device with its connected collector vessel is docked onto a sample input part of the analyser on the filter output side. Further, the transport unit is furnished with a control device for setting a maximum partial vacuum in the filter unit, the control device thus controlling the flow rate of the vacuum generating pump, i.e., a peristaltic pump.

The filter unit has on its input side (i.e., the side at which the collecting vessel containing the whole blood is attached), a suction tube and an aeration tube, which are both introduced into the collecting vessel (for instance a syringe with Luer fitting), such that no disturbing partial vacuum arises when whole blood is drawn from the collecting vessel.

To avoid the sucking-in of small air bubbles the aeration tube is introduced into the collecting vessel more deeply than the suction tube, i.e., the part of the aeration tube extending into the collecting vessel is longer than the part of the suction tube extending into the collecting vessel.

In accordance with another embodiment, a method of plasma separation from whole blood is provided comprising providing a collector vessel configured for holding whole blood; connecting the collector vessel containing whole blood to a filter unit; docking the filter unit onto the sample input part of an analyser; starting a vacuum generating pump (suction pump) of the analyser, which pump is connected to the sample input part, causing plasma to exit from the filter unit (on the side of the filter unit facing the input part of the analyser); controlling the partial vacuum in the filtering device by a control device of the analyser; and providing the plasma obtained for analyte determination in the analyser.

In accordance with one or more embodiments of the disclosure, the collector vessel containing whole blood can be connected to a filter unit by introducing a suction tube and an aeration tube of the filter unit into the collector vessel. Also, the partial vacuum in the filtering device can be controlled by a control device of the analyser, typically by pressure dependent control of the flow rate of the suction pump.

These and other features and advantages of the embodiments of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present disclosure can be best understood when read in conjunction with the following drawings, where like structure is indicated with like reference numerals and in which:

FIG. 1 is a filtering device according to an embodiment of the disclosure for separating plasma from whole blood in sectional view;

FIG. 2 is a package comprising a filtering device according to FIG. 1 and a collector vessel (syringe with Luer fitting) separately and assembled;

FIG. 3 is a system according to an embodiment of the disclosure for separating plasma from whole blood in a schematic view; and

FIGS. 4 to 6 illustrate the filtering device of FIG. 1 with differing states of the plasma front created.

Skilled artisans appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present disclosure.

### DETAILED DESCRIPTION

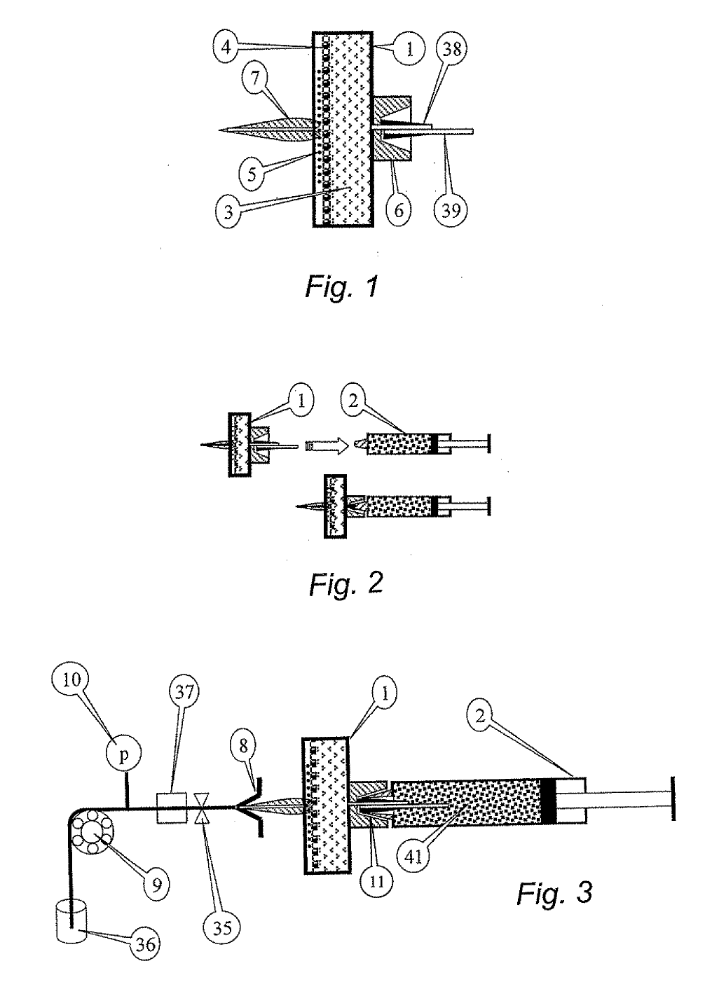

The filtering device 1 shown in FIGS. 1 to 3 for separating plasma from whole blood 41 contained in a collector vessel 2, comprises in a filter housing a layered filter, for instance consisting of a deep-bed filter 3, a stopping membrane 4 and a lateral grid 5, where on the input side a receiving element 6 with an aeration opening, e.g., an aerated Luer cone, is provided for attaching the collector vessel 2 (for instance a syringe with Luer cone), and where on the output side a capillary adapter 7 is provided for connecting to the sample input device 8 of an analyser.

The complete system schematically shown in FIG. 3 for separating plasma from whole blood is served by a transport unit of an analyser (not further shown), which has a pump 9 creating the partial vacuum required for plasma separation. The transport unit has a control device (not shown) for controlling the transport volume of the pump 9, which can be used to set a predetermined partial vacuum in the filter unit 1. For this purpose the control device has a pressure sensor 10 whose output signal is fed to the control unit of pump 9 (e.g., a peristaltic pump). The output of pump 9 is dumped into a waste container 36.

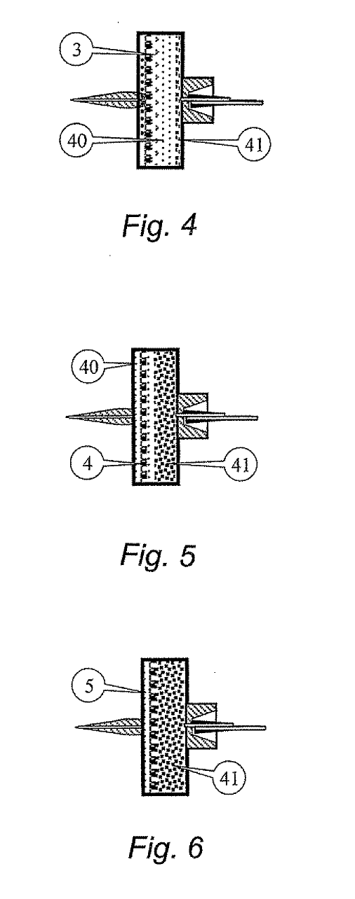

The system according to FIG. 3 may be used for plasma extraction as follows:
Taking the filter unit 1 (in detail shown in FIG. 1) from a sterile package.
Docking the receiving element 6 (i.e., aerated Luer cone) of the filter unit 1 onto a collector vessel 2 (e.g., a 2 ml syringe) containing at least 500 μl, typically 1 ml, of whole blood 41, as shown in FIG. 2.
FIG. 3: Docking the capillary adapter 7 onto the sample input device/part 8 of an analyser not further shown.
Selecting the mode “capillary measurement” on the analyser (e.g., cobas b 221 of Roche Diagnostics).
(Alternatively the output side adapter of the filter unit 1 may also be configured as a Luer fitting. In this case the mode “syringe measurement” must be selected at the analyser, followed by the docking of the filter unit 1 plus collector vessel 2.)
Suction start in the case of capillary measurement: pump 9 of the transport system of the analyser, e.g., a peristaltic pump, is activated, and in case of a peristaltic pump will start rotating, generating a partial vacuum on the output side of the filter unit 1.
(Alternatively, in mode “syringe measurement”, the pump 9 may be started manually as soon as the filter unit 1 has docked onto the sample input part 8 of the analyser.)
By means of the pressure sensor 10 of the control unit of the analyser the transport volume of pump 9 may be adjusted in such a way that a partial vacuum of not more than about 500 mbar, typically 300 mbar, more typically 100 to 150 mbar, is established at the filter unit 1.
The receiving element 6, e.g., aerated two-lumen Luer adapter (with suction tube 38 and aeration tube 39) enables pressure compensation whilst blood is sucked from the syringe. For this purpose groove-shaped channels 11 on the cone-shaped surface of the Luer adapter 6 of the filter unit 1 or a gas-permeable, typically hydrophobic layer may provide pressure compensation. The aeration tube 39 extends into the syringe somewhat farther than the suction tube 38 and thus prevents the sucking-up of incoming air bubbles, since during operation incoming air bubbles will move upwards and thus out of the suction area of the suction tube 38. At the Luer fitting of the syringe the Luer adapter 6 will seal tightly against the inner wall.
The position of the aeration tube 39 will also influence the amount of sample that can be obtained, since the end of the aeration tube 39 extending into the collector vessel will act as a “stop” for the plunger in the collector vessel.
(Alternatively, aeration of the Luer adapter 6 may also be achieved by means of porous, air-permeable plastics materials.)
The deep-bed filter 3 of the filter unit 1 may be built up from glass fibers without binding agent (typically FV-2, Whatman Inc., resp. DE 40 15 589 A1 or EP 0 239 002 A1 Boehringer-Mannheim) with a retention range of 0.5 μm to 10 μm, typically 1 μm to 5 μm, more typically <3 μm. The red blood cells (RBCs) will deposit on the thin glass fibres of the deep bed filter 3 without bursting or unduly influencing the rate of flow (see FIG. 4).
Depending on the cross-section of the filter unit 1 and on haemocrit a “plasma front” or “plasma fraction” 40 will form, which can pass the stop membrane 4 unimpededly. Residual single RBCs not retained by the deep-bed filter are filtered out by the “narrow-mesh” stop membrane 4 (FIG. 5). For this purpose the stop membrane 4 has a pore size significantly smaller than that of the deep-bed filter 3, i.e., pore diameters of less than 400 nm, typically less than 200 nm. By combining a deep-bed filter 3, which on account of its pore size already retains the greater part of blood cells, but does not impede the flow of the plasma fraction, with a subsequent stop membrane 4, which due to its smaller pore size will reliably retain remaining blood cells, but would clog swiftly on account of its limited number of pores if the preceding deep-bed filter 4 were absent, a reliable separation of blood cells without clogging of the filter can be achieved, thus making it possible to obtain a sufficiently large volume of plasma sample.
The partial vacuum established by means of the pressure sensor 10 and the controlled pump 9 together with the geometry of the filter unit will determine the flow rate and thus the shear forces acting especially on the RBCs within the stop membrane 4. Bursting of RBCs (haemolysis) is efficiently prevented by the controlled suction operation according to the present embodiment of the disclosure, with its relatively small and uniform application of a partial vacuum without large variations of pressure.
FIG. 6: The lateral grid 5 permits plasma to be collected and sucked off behind the stop membrane 4 towards the capillary adapter 7 by preventing the stop membrane 4 from “sealing off” tightly. Due to its grid structure the lateral grid 5 on the one hand acts as a non-continuous support for the stop membrane 4, letting plasma flow out on the output side of the stop membrane 4. By forming channels the grid structure furthermore enables plasma which exits over the area of the stop membrane 4, to converge towards the area of the capillary adapter 7 and to flow through the adapter into the analyser.
(This functionality of the lateral grid 5 may alternatively also be provided by stamping the bottom of the filter unit 1 or otherwise providing for sufficient roughness of its surface.)
After the desired amount of plasma has been sucked in (measured for instance by a sample sensor 35 at the entrance to the measuring chamber 37) pressure compensation is achieved by reversed operation of the peristaltic pump 9—controlled by pressure sensor 10—to avoid fractioning of the entered amount of plasma when the filter unit 1 is removed.
(Alternatively, plasma extraction may be ended when a premature pressure rise is detected by the pressure sensor 10 to avoid haemolysis and thus contamination of the extracted plasma if the haemocrit value is high and/or the sample volume is small.)
Detaching the filter unit 1 plus collector vessel 2 from the sample input part 8 of the analyser.
Positioning the sample in the analyser and analytic determination of, for instance, the haemoglobin value of the extracted plasma in the measuring chamber 37 (e.g., an oxymeter).

Duration of the suction phase, desired sample volume and the dimensions of the filter are interdependent of each other and may be chosen by the expert in such a way that plasma extraction according to the method of the disclosure may be carried out without additional haemolysis.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A system for separating plasma from whole blood, comprising:
a collector vessel for receiving whole blood,
a filtering device for extracting plasma, whose input side is attachable to the collector vessel, and
a transport unit furnished with a pump for creating a partial vacuum, wherein
the transport unit is part of an analyser and the filtering device together with the attached collector vessel may be docked onto the sample input part of the analyser via the output side of the filtering device, and
the transport unit is provided with a control device for controlling the flow rate of the vacuum generating pump in order to set the highest permissible partial vacuum in the filtering device.

2. The system according to claim 1, wherein said control device has a pressure sensor whose output signal is fed to the control unit of the vacuum generating pump.

3. The system according to claim 1, wherein the filtering device further comprises a suction tube and an aeration tube which may be together introduced into the collector vessel.

4. The system according to claim 3, wherein the aeration tube extends farther into the collector vessel than the suction tube.

5. The system according to claim 1, wherein the filtering device on its input side has a docking site with aeration openings for the collector vessel, and on its output side has a capillary adapter for connecting to the sample input part of an analyser.

6. The system according to claim 5, wherein the docking site is an aerated Luer cone.

7. The system according to claim 1, wherein the filtering device further comprises a layered filter in a filter housing.

8. The system according to claim 7, wherein said filtering device comprises a deep-bed filter, a stop membrane and a lateral grid.

9. A method for separating plasma from whole blood, comprising:
providing a collector vessel configured for holding whole blood;
connecting the collector vessel containing whole blood to a filtering device, preferably by introducing a suction tube and an aeration tube of the filtering device into the collector vessel;
docking the filtering device onto the sample input part of an analyser;
starting a vacuum generating pump of the analyser, which pump is connected to the sample input part, causing plasma to exit from the filtering device;
controlling the partial vacuum in the filtering device by a control device of the analyser, preferably by pressure dependent control of the flow rate of the suction pump; and
providing the plasma obtained for analyte determination in the analyser.

10. The method according to claim 9, wherein connecting the collector vessel containing whole blood to a filtering device includes introducing a suction tube and an aeration tube of the filtering device into the collector vessel.

11. The method according to claim 9, wherein controlling the partial vacuum in the filtering device by a control device of the analyser includes pressure dependent control of the flow rate of the suction pump.

12. The method according to claim 9, wherein a partial vacuum of <500 mbar is established in the filtering device.

13. The method according to claim 9, wherein a partial vacuum of <300 mbar is established in the filtering device.

14. The method according to claim 9, wherein a partial vacuum of between 100 and 150 mbar is established in the filtering device.

15. The method according to claim 9, wherein the established partial vacuum is monitored by a pressure sensor of the analyser.

16. The method according to claim 9, wherein plasma extraction is automatically ended by a premature pressure rise in the filtering device to avoid haemolysis.
